(19) United States
(12) Patent Application Publication
(10) Pub. No.: US 2019/0309257 A1
(43) Pub. Date: Oct. 10, 2019

(54) METHOD FOR PRODUCING CELL LOCATING PLATE, METHOD FOR PRODUCING MEMBER FOR CONTAINING CELLS, MEMBER FOR CONTAINING CELLS, AND CELL LOCATING PLATE

(71) Applicants: Tomoyuki Aratani, Kanagawa (JP); Chihiro Kubo, Kanagawa (JP); Hidekazu Yaginuma, Kanagawa (JP); Naoki Satoh, Kanagawa (JP)

(72) Inventors: Tomoyuki Aratani, Kanagawa (JP); Chihiro Kubo, Kanagawa (JP); Hidekazu Yaginuma, Kanagawa (JP); Naoki Satoh, Kanagawa (JP)

(21) Appl. No.: 16/375,490

(22) Filed: Apr. 4, 2019

(30) Foreign Application Priority Data
Apr. 6, 2018 (JP) 2018-074046
Mar. 5, 2019 (JP) 2019-039295

(51) Int. Cl. C12N 5/00; C12M 1/12; C12N 5/071

(52) U.S. Cl. CPC C12N 5/0068; C12N 2533/30; C12N 5/0671; C12M 25/14

(57) ABSTRACT

Provided is a method for producing a cell locating plate, including: a step of laminating a structural layer over a plane of a base material, the structural layer being a layer that is formed of a cell non-adhesive material and in which a plurality of holes are formed; a step of coating a coating liquid in the holes and over the plane of the base material exposed through the holes; and a step of discharging a cell suspension onto the plane coated with the coating liquid.

### CROSS-REFERENCE TO RELATED APPLICATIONS

The present application claims priority under 35 U.S.C. § 119 to Japanese Patent Application No. 2018-074046 filed Apr. 6, 2018 and Japanese Patent Application No. 2019-039295 filed Mar. 5, 2019. The contents of which are incorporated herein by reference in their entirety.

### BACKGROUND OF THE INVENTION

### Field of the Invention

The present disclosure relates to a method for producing a cell locating plate, a method for producing a member for containing cells, a member for containing cells, and a cell locating plate.

### Description of the Related Art

In recent years, along with the evolution of the stem cell technologies, techniques for artificially forming tissues using a plurality of cells have been being developed. In artificial formation of tissues, a technique for locating cells in an optional manner is indispensable. Hence, various attempts have been already made, and, for example, cell sheet methods, spheroid lamination methods, gel extrusion methods, and inkjet methods are known.

Among these methods, cell locating techniques by inkjet methods are excellent in controlling a plurality of kinds of cells to arbitrary positions in arbitrary numbers of cells, and are considered to be one of promising techniques capable of artificially forming tissues. Therefore, various proposals have been made.

For example, in order to form a micro-pattern of cells over a substrate, there has been proposed a cell immobilized substrate obtained by locating a cell adhesive material in a pattern over a substrate by an inkjet method (for example, see Japanese Unexamined Patent Application Publication Nos. 2012-187072 and 2009-131240).

Further, in order to prevent elution of tackifier layer-constituting materials used over a cell culture substrate, there has been proposed a cell culture substrate including a tackifier layer, an adhesive layer, a cell culture layer, and a protective layer over a substrate, wherein an exposed surface of the tackifier layer is covered with the protective layer (for example, see Japanese Unexamined Patent Application Publication No. 2013-099271).

Furthermore, as a technique for precisely locating a cell assembly over a cell adhesive surface, there has been disclosed a cell assembly forming tool including wells formed of a first substrate in which through holes are formed and a cell non-adhesive second substrate, and configured for cells to be seeded into each well and formed as one cell assembly, in order that the cell assembly may be subsequently transferred onto a cell-adhesive surface (see Japanese Unexamined Patent Application Publication No. 2010-233456).

### SUMMARY OF THE INVENTION

According to one aspect of the present disclosure, a method for producing a cell locating plate includes a step of laminating a structural layer over a plane of a base material, the structural layer being a layer that is formed of a cell non-adhesive material and in which a plurality of holes are formed, a step of coating a coating liquid in the holes and over the plane of the base material exposed through the holes, and a step of discharging a cell suspension onto the plane coated with the coating liquid.

### BRIEF DESCRIPTION OF THE DRAWINGS

FIG. 1A is a view illustrating an example of a state before holes are formed in a structural layer by laser machining;

FIG. 1B is a view illustrating an example of a state of a structural layer, in which holes are formed, being located over a base material;

FIG. 1C is a view illustrating another example of a state of a structural layer, in which holes are formed, being disposed over a base material;

FIG. 2 is a schematic cross-sectional view illustrating an example of a liquid droplet forming device;

FIG. 3 is a graph plotting an example of a discharging signal and a suppressing signal;

FIG. 4A is a view illustrating an example of an operation of a liquid droplet forming device;

FIG. 4B is a view illustrating an example of an operation of a liquid droplet forming device;

FIG. 4C is a view illustrating an example of an operation of a liquid droplet forming device;

FIG. 5A is a graph plotting another example of a discharging signal and a suppressing signal;

FIG. 5B is a graph plotting another example of a discharging signal and a suppressing signal;

FIG. 6 is a schematic view illustrating an example of a cell contained member producing apparatus mounted with a plurality of liquid droplet forming devices;

FIG. 7 is a view illustrating an example of an operation for discharging a cell adhesive material ink;

FIG. 8A is a view illustrating an example of an operation for discharging a coating liquid;

FIG. 8B is a view illustrating an example of an operation for discharging a coating liquid;

FIG. 9A is a view illustrating an example of an operation for discharging a cell ink;

FIG. 9B is a view illustrating an example of an operation for discharging a cell ink;

FIG. 10 is a view illustrating an example of an operation for discharging a cell ink by an existing inkjet method;

FIG. 11A is an image illustrating holes formed by laser-machining a structural layer in Example 1;

FIG. 11B is an image illustrating holes formed by laser-machining a structural layer in Example 1;

FIG. 12A is an image illustrating holes formed by laser-machining a structural layer in Example 2;

FIG. 12B is an image illustrating holes formed by laser-machining a structural layer in Example 2;

FIG. 13 is a view illustrating an operation for discharging a cell adhesive material ink in Example 7;

FIG. 14 is a view illustrating an operation for discharging a cell ink in Example 8; and

FIG. 15 is an image of a cell ink located in a state of being stained into two kinds in Example 9.

### DESCRIPTION OF THE EMBODIMENTS

### (Member for Containing Cells)

A member for containing cells of the present disclosure includes a base material having at least a plane, a structural layer over the plane, the structural layer being formed of a cell non-adhesive material and including a plurality of holes, and a coating liquid in the holes, the coating liquid coating the plane of the base material exposed through the holes, and further includes other members as needed. In other words, the member for containing cells of the present disclosure is produced by laminating a structural layer, which is obtained by forming a plurality of holes in a layer formed of a cell non-adhesive material, over a plane of a base material having at least the plane, and adding a coating liquid for coating the plane of the base material exposed through the holes, in the holes. The member for containing cells further includes other members as needed.

The member for containing cells of the present disclosure is based on a problem found by the present inventors from existing techniques.

For example, the existing cell immobilized substrate obtained by locating a cell adhesive material in a pattern over a substrate by an inkjet method has a problem that cells cannot be contained dividedly in holes and cells may possibly coalesce with each other because the cell adhesive material is merely patterned over the substrate by an inkjet method.

Further, the existing cell culture substrate has a problem that cells discharged by an inkjet method tend to be damaged when landing, because cells are directly landed over the cell adhesive layer.

Furthermore, the existing cell assembly forming tool, which is based on seeding of cells, has difficulty producing a tissue, because it is difficult to locate a plurality of kinds of cells at desired positions by seeding.

With the structural layer including a plurality of holes over a base material, the member for containing cells of the present disclosure can contain cells dividedly in the plurality of holes when a cell suspension is discharged into the holes, making it possible to suppress mislocation of cells due to coalescing of landed liquid droplets.

Furthermore, by containing the landed cell suspension in the holes, the member for containing cells of the present disclosure can reduce the surface area of the cell suspension contacting the atmosphere, making it possible to suppress drying of the cells.

Moreover, by including the coating liquid coating the plane of the base material exposed through the holes in the holes, the member for containing cells of the present disclosure can suppress cells discharged by an inkjet method from being damaged when landing, and can suppress drying of the cells.

The member for containing cells of the present disclosure is suitably used for containing a cell suspension containing cells in the holes.

### <Cell Suspension>

The cell suspension is not particularly limited and may be appropriately selected depending on the intended purpose so long as the cell suspension contains cells. For example, the cell suspension contains cells to be patterned, and a dispersion medium in which cells are dispersed, and further contains other components as needed.

The “cell suspension” may be referred to as “cell ink” or simply “ink”.

### <<Cells>>

For example, the kind of the cells is not particularly limited and may be appropriately selected depending on the intended purpose. Taxonomically all kinds of cells can be used regardless of whether the cells are eukaryotic cells, prokaryotic cells, multicellular organism cells, and unicellular organism cells. One of these kinds of cells may be used alone or two or more of these kinds of cells may be used in combination.

Examples of the eukaryotic cells include animal cells, insect cells, plant cells, and fungi. One of these kinds of eukaryotic cells may be used alone or two or more of these kinds of eukaryotic cells may be used in combination. Among these eukaryotic cells, animal cells are preferable. When a cell assembly is formed with the cells, adherent cells having cell adhesiveness of a level at which cells adhere with each other and are not isolated without a physicochemical treatment are more preferable.

Adherent cells are not particularly limited and may be appropriately selected depending on the intended purpose. Examples of adherent cells include differentiated cells and undifferentiated cells. One of these kinds of adherent cells may be used alone or two or more of these kinds of adherent cells may be used in combination.

Examples of differentiated cells include: hepatocytes, which are parenchymal cells of a liver; stellate cells; Kupffer cells; endothelial cells such as vascular endothelial cells, sinusoidal endothelial cells, and corneal endothelial cells; fibroblasts; osteoblasts; osteoclasts; periodontal ligament-derived cells; epidermal cells such as epidermal keratinocytes; epithelial cells such as tracheal epithelial cells, intestinal epithelial cells, cervical epithelial cells, and corneal epithelial cells; mammary glandular cells; pericytes; muscle cells such as smooth muscle cells and myocardial cells; renal cells; pancreatic islet cells; nerve cells such as peripheral nerve cells and optic nerve cells; chondrocytes; and bone cells. Adherent cells may be primary cells directly taken from tissues or organs, or may be cells obtained by passaging primary cells a few times. One of these kinds of cells may be used alone or two or more of these kinds of cells may be used in combination.

Undifferentiated cells are not particularly limited and may be appropriately selected depending on the intended purpose. Examples of undifferentiated cells include: pluripotent stein cells such as embryotic stem cells, which are undifferentiated cells, and mesenchymal stem cells having pluripotency; unipotent stem cells such as vascular endothelial progenitor cells having unipotency; and iPS cells. One of these kinds of undifferentiated cells may be used alone or two or more of these kinds of undifferentiated cells may be used in combination.

Examples of the prokaryotic cells include eubacteria and archaea.

As the dispersion medium, a culture medium for cell culture or a buffer is preferable, like the coating liquid described below. Examples of the dispersion medium include phosphate buffered saline.

The other components are not particularly limited and may be appropriately selected depending on the intended purpose. Examples of the other components include a humectant, an intercellular distance adjusting material, a dispersant, and a pH adjustor.

The humectant is not particularly limited and may be appropriately selected depending on the intended purpose. Examples of the humectant include gelatinous polysaccharides.

The gelatinous polysaccharides are not particularly limited and may be appropriately selected depending on the intended purpose. Examples of the gelatinous polysaccharides include calcium alginate, gellan gum, agarose, guar gum, xanthan gum, carrageenan, pectin, locust bean gum, Tamarind gum, diutan gum, and carboxymethyl cellulose. One of these gelatinous polysaccharides may be used alone or two or more of these gelatinous polysaccharides may be used in combination. Among these gelatinous polysaccharides, calcium alginate is preferable.

Calcium alginate is a salt in which calcium ion is bonded with carboxyl group of alginic acid. Calcium ion is divalent. Hence, calcium ion is bonded (ionically cross-linked) with two carboxyl groups in a manner to bridge the two carboxyl groups, to thicken the viscosity. In this way, calcium alginate can suppress drying of the cell ink. Here, calcium ion is contained in a dispersion medium, and is considered to bond with calcium ion that has become excessive due to drying-induced concentration. Therefore, a function as an osmotic pressure adjustor can also be expected.

The intercellular distance adjusting material is not particularly limited and may be appropriately selected depending on the intended purpose. An intercellular distance adjusting material having biocompatibility is preferable.

### <Discharging>

As a unit configured to discharge a liquid droplet of the cell ink onto the base material, a liquid droplet discharging unit of an inkjet type is preferable.

Examples of the liquid droplet discharging unit of the inkjet type include a so-called piezo type (for example, see Japanese Examined Patent Publication No. 02-51734) using a piezoelectric element as a pressure generating unit to pressurize the cell ink to change the volume of the cell ink and discharge liquid droplets, a so-called thermal type (for example, see Japanese Examined Patent Publication No. 61-59911) using a heating resistor to heat the cell ink and generate bubbles, and an electrostatic type (for example, see Japanese Unexamined Patent Application Publication No. 06-71882) using a vibration plate and an electrode disposed counter to the vibration plate to deform the vibration plate by an electrostatic force generated between the vibration plate and the electrode and change the volume of the cell ink to discharge liquid droplets.

A discharging speed is not particularly limited, may be appropriately selected, and is preferably 0.1 m/s or higher but 10 m/s or lower. When the discharging speed is 0.1 m/s or higher but 10 m/s or lower, it is possible to suppress cells from being damaged when landing, so long as the minimum thickness of the coating liquid in the holes is at least 5 micrometers.

A method for obtaining the discharging speed is not particularly limited and may be appropriately selected depending on the intended purpose. Examples of the method include a calculation method based on a temporal interval between flames of captured images and a travelling distance of an ink liquid droplet, in the case of capturing images of a flying ink liquid droplet in order to count the number of cells in the ink liquid droplet.

The number of cells contained in a liquid formed by one discharging is not particularly limited, may be appropriately selected, and is preferably 1 or more but 2 or less. When the number of cells contained in a liquid droplet formed by one discharging is 1 or more but 2 or less, a discharging failure due to clogging of a nozzle of an inkjet head with cells is less likely to occur. There is another advantage that low variation in the number of cells also suppresses variation in the cell concentration from hole to hole.

### <Base Material>

The shape of the base material is not particularly limited and may be appropriately selected depending on the intended purpose so long as the base material has at least a plane. A planar shape is preferable. When the shape of the base material is a planar shape, i.e., when the base material is a substrate, there is an advantage in terms of easy machinability.

Here, a plane refers to flatness in a broad sense, with a degree of flatness enough to prevent leakage of a liquid when the liquid is injected into the holes in the structural layer when the structural layer including the holes is laminated over the plane. The plane may have bosses and dents microscopically. The range of the plane is not particularly limited and may be appropriately selected depending on the intended purpose. It is preferable that a range corresponding to the bottoms of the holes be planar.

The structure of the base material is not particularly limited and may be appropriately selected depending on the intended purpose so long as the base material includes the structural layer described below.

The size of the base material is not particularly limited and may be appropriately selected depending on the intended purpose.

The constituent material of the base material is not particularly limited and may be appropriately selected depending on the intended purpose so long as the constituent material is a cell non-adhesive material. Examples of the constituent material include organic materials and inorganic materials described below. One of these constituent materials may be used alone or two or more of these constituent materials may be used in combination. Among these constituent materials, a constituent material to which a cell adhesive material is easily absorbable is preferable. When a cell adhesive material is easily absorbable to the constituent material of the base material, the cell adhesive material can adhere to the base material in a stable state when the cell adhesive material is discharged onto the base material corresponding to the bottoms of the holes.

### —Cell Non-Adhesive Material—

Cell non-adhesiveness refers to a lower adhesiveness with intended cells than at least the adhesiveness of the cell adhesive material to be used.

A method for measuring cell non-adhesiveness is not particularly limited and may be appropriately selected depending on the intended purpose. Examples of the method include a method of measuring and evaluating adhesiveness of cells with a material by inserting a needle-like AFM probe into cells cultured over the material and lifting the probe to peel the cells from the substrate to measure a load applied on the probe by AFM. As another method, for example, there is a simple method of flowing, for example, pure water over cells cultured over the material, and evaluating cell non-adhesiveness by adhesion peeling rates of the cells from the material before and after flowing the pure water.

The cell non-adhesive material is not particularly limited and may be appropriately selected depending on the intended purpose. A water-repellent material is preferable. When the cell non-adhesive material is a water-repellent material, there is an advantage that the cell non-adhesive material is more difficult for cells to adhere.

The cell non-adhesive material is not particularly limited and may be appropriately selected depending on the intended purpose. A silicon-containing material is preferable.

The silicon-containing material is not particularly limited and may be appropriately selected depending on the intended purpose. In terms of biocompatibility, polydimethyl siloxane (PDMS) is preferable.

The organic materials are not particularly limited and may be appropriately selected depending on the intended purpose. Examples of the organic materials include polyethylene terephthalate (PET), polystyrene (PS), polycarbonate (PC), TAC (triacetyl cellulose), polyimide (PI), nylon (Ny), low density polyethylene (LDPE), medium density polyethylene (MDPE), vinyl chloride, vinylidene chloride, polyphenylene sulfide, polyether sulfone, polyethylene naphthalate, polypropylene, acrylic-based materials such as urethane acrylate, cellulose, and silicone-based materials such as polydimethyl siloxane (PDMS).

The inorganic materials are not particularly limited and may be appropriately selected depending on the intended purpose. Examples of the inorganic materials include glass and ceramics.

Examples of the cell adhesive material include a protein selected from the extracellular matrix.

Examples of the protein selected from the extracellular matrix include fibronectin, laminin, tenascin, vitronectin, RGD (arginylglycylaspartic acid) sequence-containing peptides, YIGSR (tyrosine-isoleucine-glycine-serine-arginine) sequence-containing peptides, collagen, atelocollagen, and gelatin. Additional examples of the protein selected from the extracellular matrix include mixtures of the materials described above, matrigel, Pura Matrix, and fibrin. Among these proteins, collagen is preferable. There are many kinds of collagens. Collagens are known to thicken depending on the concentration or the temperature. By adding collagen in the cell ink, it is possible to cause the cell ink to thicken when the concentration of the cell ink increases. Further examples of the protein selected from the extracellular matrix include basic polymers such as polylysine and basic compounds such as aminopropyl triethoxysilane.

In the case of discharging the cell adhesive material, it is possible to discharge the cell adhesive material in the form of a solution containing the cell adhesive material. In this case, the solution may contain biocompatible particles.

The biocompatible particles are not particularly limited and may be appropriately selected so long as the biocompatible particles have compatibility with living organisms such as cells. Examples of the biocompatible particles include gelatin particles and collagen particles. One of these kinds of particles may be used alone or two or more of these kinds of particles may be used in combination.

When the biocompatible particles are gelatin particles, gelatin as the raw material of the gelatin particles is not particularly limited and may be appropriately selected depending on the intended purpose. Examples of the gelatin include a product named: APH-250 (available from Nitta Gelatin Inc.).

The gelatin particles having a particulate shape can improve adhesiveness of cells with the base material, and can be present in a tissue without being degraded by the cells for a longer time than gelatin having a non-particulate shape. Therefore, there are advantages that the gelatin particles can improve adhesiveness of cells and are used as a source of nutrients for the cells for a long term.

It is preferable that the biocompatible particles be cross-linked by a cross-linking agent in the structure. By cross-linking by a cross-linking agent, the cumulant diameter of the biocompatible particles can be made small and cell proliferation can be promoted in the solution containing the cell adhesive material.

The cross-linking agent is not particularly limited and may be appropriately selected depending on the intended purpose. Examples of the cross-linking agent include: aldehydes such as glutaraldehyde and formaldehyde; glycidyl ethers such as ethylene propylene diglycidyl ether, glycerol polyglycidyl ether, diglycerol polyglycidyl ether, sorbitol polyglycidyl ether, and ethylene glycol diglycidyl ether; isocyanates such as hexamethylene diisocyanate, α-tolidine isocyanate, tolylene diisocyanate, naphthylene-1,5-diisocyanate, 4,4-diphenylmethane diisocyanate, and triphenylmethane-4,4,4-triisocyanate; calcium gluconate; methyl (1S,2R,6S)-2-hydroxy-9-(hydroxymethyl)-3-oxabicyclo [4.3.0] nona-4,8-diene-5-carboxylate (genipin); combination of polyphenol and an oxidant such as horseradish peroxidase; and a compound containing a succinimide group. One of these cross-linking agents may be used alone or two or more of these cross-linking agents may be used in combination. Among these cross-linking agents, aldehydes are preferable and glutaraldehyde is more preferable.

The content of the cross-linking agent is preferably 1% by mass or greater but 20% by mass or less and more preferably 2% by mass or greater but 10% by mass or less relative to the total amount of the raw material of the biocompatible particles. When the content of the cross-linking agent is 1% by mass or greater but 20% by mass or less, the cumulant diameter of the biocompatible particles can be made small and cell proliferation can be promoted in the solution containing the cell adhesive material.

The content of the biocompatible particles is preferably 0.5% by mass or greater but 10% by mass or less and more preferably 1% by mass or greater but 5% by mass or less relative to the total amount of the solution containing the cell adhesive material. When the content of the biocompatible particles is 0.5% by mass or greater but 10% by mass or less, cell proliferation can be promoted, with sufficient adhesion of cells with a tissue, which is a three-dimensional cell assembly.

The range in which the cell adhesive material is attached is not particularly limited and may be appropriately selected depending on the intended purpose. Examples of the range include the whole area or a partial area of the bottoms (base material) of the holes. About the center of the bottoms is preferable. When the range in which the cell adhesive material is attached is about the center of the bottoms, it becomes harder for cells to adhere to the wall surfaces (side surfaces) of the holes, making control on the number of cells to be formed over the base material highly accurate.

### —Preparation Example of Sample Liquid Containing Cell Adhesive Material—

The biocompatible particles are dispersed in pure water obtained with a pure water producing apparatus (product name: GSH-2000, available from ADVANTEC Co., Ltd.), at a concentration of 0.5% by mass. The liquid amount for measurement is 5 mL. The biocompatible particles are subjected to dispersion treatment by stirring with a stirrer including a 20 mm rotor, with stirring kept for about one day at 200 rpm. In this way, the sample liquid can be prepared.

### —Measurement Conditions—

Solvent: water (refractive index: 1.3314, viscosity at 25 degrees C.: 0.884 mPa·s (cP), with appropriate setting of the optimum light volume adjustment by an ND filter)
Measuring probe: a probe for a concentrated system
Measurement routine: measurement at 25 degrees C. for 180 seconds, then measurement at 25 degrees C. for 600 seconds (monitoring of the change of the particle diameter during gradual change of the liquid temperature from 25 degrees C. to 35 degrees C. started in response to temperature change to 35 degrees C. on the main body side), and then measurement at 35 degrees C. for 180 seconds

### <<Structural Layer>>

The shape of the structural layer is not particularly limited and may be appropriately selected depending on the intended purpose so long as the structure layer includes at least any one of a plurality of holes.

The average thickness of the structural layer is not particularly limited, may be appropriately selected depending on the intended purpose, and is preferably 10 micrometers or greater. When the average thickness of the structural layer is 10 micrometers or greater, there are advantages that a capacity for receiving the cell ink can be secured, and that the structural layer can be easily handled when laminated over the substrate.

The structure of the structural layer is not particularly limited and may be appropriately selected depending on the intended purpose. Examples of the structure of the structural layer include a single-layer structure and a laminated structure. Examples of the laminated structure include a structure with a masking tape laminated. In this case, the masking tape is not particularly limited and may be appropriately selected depending on the intended purpose. A masking tape having a thin tackifier layer and a weak tackifying force is preferable. When a masking tape has a thin tackifier layer and a weak tackifying force, there is an advantage that when the laminated masking tape is machined with a laser to open holes, the wall surfaces of the holes are less likely to be sticky with the tackifier layer. The material of the masking tape is not particularly limited and may be appropriately selected depending on the intended purpose. Examples of the material of the masking tape include polyethylene.

The size of the structural layer is not particularly limited and may be appropriately selected depending on the intended purpose. Examples of the size of the structural layer include the same size as the size of the base material.

The constituent material of the structural layer is not particularly limited and may be appropriately selected depending on the intended purpose. Examples of the constituent material of the structural layer include the materials raised as examples of the constituent material of the base material. One of these constituent materials may be used alone or two or more of these constituent materials may be used in combination. Among these constituent materials, a cell non-adhesive material is preferable, and a material that can be easily peeled from the base material is more preferable in the case of producing a three-dimensional tissue. The constituent material of the structural layer may be the same as or different from the constituent material of the base material.

### —Holes—

The holes are not particularly limited and may be appropriately selected depending on the intended purpose so long as the holes have a through hole shape.

The shape of the holes in a plan-view perspective is not particularly limited and may be appropriately selected depending on the intended purpose. Examples of the shape of the holes include a circular shape, an elliptic shape, a triangular shape, and a quadrangular shape.

The structure of the wall surfaces (side surfaces) of the holes is not particularly limited and may be appropriately selected depending on the intended purpose. Examples of the structure of the wall surfaces of the holes include a cylindrical shape, a tapered shape, and a bossed-dented shape.

The method for forming the holes is not particularly limited and may be appropriately selected depending on the intended purpose. Examples of the method include laser machining, photolithographic machining, drill machining, and molding with a molding die. Among these methods, laser machining is preferable. When the method for forming the holes is laser machining, there are advantages that a pattern that enables formation of a desired tissue can be formed easily and minutely, and that machining without a direct contact with the structural layer makes it easier to secure biocompatibility.

The locations of the plurality of holes are not particularly limited and may be appropriately selected depending on the intended purpose. For example, the locations of the plurality of holes may coincide with the pattern that enables formation of a desired tissue.

The distance between the centers of mutually adjacent holes, i.e., the pitch interval between the holes is preferably 300 micrometers or less. When the pitch interval between the holes is 300 micrometers or less, there is an advantage that landed cells are less likely to coalesce with each other, making it possible to maintain the accuracy of the cell discharging positions.

The plane of the base material exposed through the holes in the structural layer serves as a surface onto which ink liquid droplets discharged by an inkjet method land.

It is preferable to sterilize the base material and the structural layer because the cell ink will contact the base material and the structural layer.

### <Coating Liquid>

The coating liquid is not particularly limited and may be appropriately selected depending on the intended purpose so long as the coating liquid can coat the plane of the base material exposed through the holes in the holes. Preferable examples of the coating liquid include a culture medium for cell culture and a buffer.

The culture medium is a solution that contains components needed for formation and sustainment of a tissue, prevents drying, and adjusts the external environment such as the osmotic pressure. Any substance may be appropriately selected and used as the culture medium, so long as the substance is known as a culture medium. When there is no need for immersing the cells in the culture medium liquid all the time, the culture medium may be appropriately removed from the cell ink.

The buffer is for adjusting pH depending on the cells and the intended purpose. A known buffer may be appropriately selected and used.

The viscosity of the coating liquid is not particularly limited and may be appropriately selected depending on the intended purpose. It is preferable that the viscosity of the coating liquid be rather high. When the viscosity of the coating liquid is high, there are advantages that it is easier to suppress cells from being damaged when landing, and that it is easier to suppress drying of the cells.

The minimum thickness of the coating liquid is not particularly limited, may be appropriately selected depending on the intended purpose, and is preferably 5 micrometers or greater. When the minimum thickness of the coating liquid is 5 micrometers or greater, there is an advantage that the death rate of cells when the cell ink lands can be reduced.

Examples of the method for obtaining the minimum thickness of the coating liquid include a method of determining the minimum value among the measured values of the thickness of the coating liquid obtained at random ten positions with a spectral-interference laser displacement meter (SI-F01, available from Keyence Corporation), as the minimum thickness.

Examples of a method for injecting the coating liquid into the holes include a method of seeding the coating liquid and a method of injecting the coating liquid by an inkjet method. Of these methods, the injecting method by an inkjet method is preferable. With the injecting method by an inkjet method, there is an advantage that variation in the liquid amount of the coating liquid to be injected into the holes can be suppressed.

It has been described that the structural layer includes holes having a through hole shape. This is non-limiting. The structural layer may include holes having a one-end-opened bottomed shape. In other words, the member for containing cells of the present disclosure may be obtained by forming a plurality of cells in a layer formed of a cell non-adhesive material and adding a coating liquid for coating the bottoms of the holes into the holes. In this case, the coating liquid for coating the bottoms of the holes are contained in the holes. The bottoms of the holes in the structural layer serve as a surface onto which ink liquid droplets discharged by an inkjet method land. The structural layer may be omitted and holes may be formed in the base material.

### (Method for Producing Member for Containing Cells)

A method for producing a member for containing cells includes a step of laminating a structural layer over a plane of a base material, the structural layer being a layer that is formed of a cell non-adhesive material and in which a plurality of holes are formed, a step of attaching a cell adhesive material in the holes, and a step of coating a coating liquid in the holes and over the plane of the base material exposed through the holes.

### (Method for Producing Cell Locating Plate)

A method for producing a cell locating plate includes a step of laminating a structural layer over a plane of a base material, the structural layer being a layer that is formed of a cell non-adhesive material and in which a plurality of holes are formed, a step of coating a coating liquid in the holes and over the plane of the base material exposed through the holes, and a step of discharging a cell suspension onto the plane coated with the coating liquid. The method for producing a cell locating plate may further include a step of attaching a cell adhesive material in the holes.

### <Method for Producing Member for Containing Cells>

### [Formation of Holes in Structural Layer]

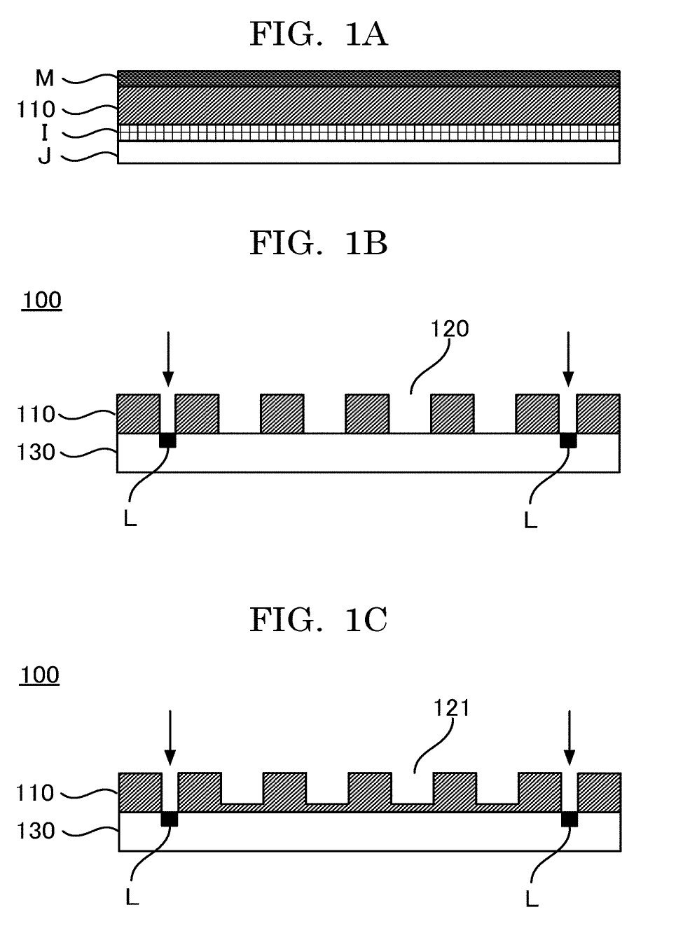

FIG. 1A is a view illustrating an example of a state before holes are formed in the structural layer by laser machining.

As illustrated in FIG. 1A, pasting of, for example, a mask film M over a surface of a structural layer 110 at a laser incident side makes it possible to suppress machining debris that may occur during laser machining, making it possible to improve the cell survival rate during cell culture. Further, use of the mask film M having a higher laser absorbency than the structural layer 110 makes it possible to improve machinability, enabling more minute machining and improvement in the cell resolution.

In the case of forming the structural layer 110 with an organic material, after the organic material is coated over a jig J with a secured flatness, such as glass in order to suppress variation in the height of holes 120, the organic material is subjected to, for example, thermal curing, to have a bulk. When glass is used as the jig J and PDMS excellent in biocompatibility is used as the structural layer 110, it is preferable to provide a buffer material I such as a polyimide-based material between the jig J and the structural layer 110, in order to prevent thermal adhesion between the jig J and the structural layer 110.

### [Disposition of Structural Layer Over Base Material]

FIG. 1B is a view illustrating an example of a state of the structural layer, in which holes are formed, being disposed over the base material.

As illustrated in FIG. 1B, when there is a need for positional alignment of the structural layer 110 with a base material 130, alignment marks L are formed on the base material 130 and the structural layer 110 for positional alignment. In this case, use of, for example, a camera enables a highly accurate positional alignment. Also in the case of a three-dimensional tissue, positional alignment of each structural layer 110 with the base material 130 enables the holes 120 (cells) to be aligned in place.

When the holes having a through hole shape formed in the structural layer are changed to holes having a one-end-opened bottomed shape, the holes are as illustrated in FIG. 1C.

### (Cell Contained Member)

A cell contained member of the present disclosure is the above-described member for containing cells in which cells are contained.

The cell contained member of the present disclosure includes the member for containing cells described above, and cells located in the holes or openings in the member for containing cells, and further includes other members as needed. In other words, the cell contained member is obtained by discharging cells into the holes or openings in the member for containing cells by an inkjet method, with other steps as needed.

Cells to be contained are not particularly limited and may be appropriately selected depending on the intended purpose. It is preferable to locate two or more kinds of cells in the holes or openings.

The cell adhesive material may be previously discharged into the holes.

It is preferable that the cell contained member be in the form of a cell locating plate.

Here, a cell locating plate refers to a plate in which it is known what kinds of cells are contained in which holes. When the cell contained member is a cell locating plate, there is an advantage that screening with a predetermined chemical does not depend on the skill of the experimenter, making it more likely to obtain results of experiment conditions with little variation. Furthermore, because a cell locating plate can be made small in size compared with typical wells, it is possible to save the amount of the predetermined chemical used during the screening.

A cell culture time is not particularly limited, may be appropriately selected depending on the intended purpose, and is preferably 24 hours or longer in terms of bringing the cells into close adhesion with the cell adhesive material.

### <Other Members>

Other members are not particularly limited and may be appropriately selected depending on the intended purpose. A protective cover configured to cover the member for containing cells and the cell contained member is preferable. With the protective cover, there is an advantage that contamination can be suppressed and stacking is available.

Examples of the protective cover include a sheet-shaped cover and a lid configured to tightly close a hole.

### [Method for Producing Cell Contained Member]

A method for producing the cell contained member will be described below with reference to the drawings. The same components will be denoted by the same reference numerals throughout the drawings, and redundant description about the same components may be skipped.

### <Liquid Droplet Forming Device>

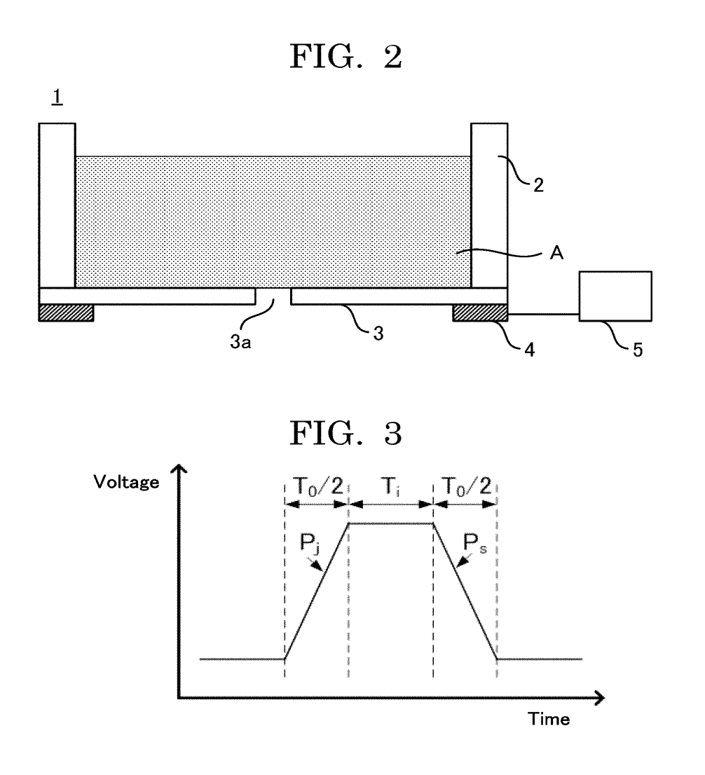

FIG. 2 is a schematic cross-sectional view illustrating an example of a liquid droplet forming device. In the following description, “liquid droplet forming device” may be referred to as “inkjet head”.

As illustrated in FIG. 2, a liquid droplet forming device 1 includes a liquid chamber 2 configured to contain a liquid, a membrane 3 in which a discharging hole (nozzle) 3a is formed, a piezoelectric element 4, and a driving unit 5 configured to output a driving signal to the piezoelectric element 4.

In the present embodiment, for expediency, a side of the liquid chamber 2 having the liquid surface is referred to as upper side, and a side of the liquid chamber 2 having the piezoelectric element 4 is referred to as lower side. Further, a surface of each portion at a side at which the liquid chamber 2 is present is referred to as upper surface, and a surface of each portion at a side at which the piezoelectric element 4 is present is referred to as lower surface.

The liquid chamber 2 includes the membrane 3 at the bottom, and can contain a cell ink A.

The cell ink A is not particularly limited and may be appropriately selected depending on the intended purpose.

Examples of the material of the liquid chamber 2 include metals, silicon, and ceramics.

The size of the liquid chamber 2 is not particularly limited and may be appropriately selected depending on the intended purpose.

The amount of the cell ink A that can be contained in the liquid chamber 2 is not particularly limited, may be appropriately selected depending on the intended purpose, and may be from 1 microliter through 1 mL, and may be from 1 microliter through 50 microliters when the cell ink A is a cell suspension in which cells are dispersed.

The membrane 3 is disposed as the bottom of the liquid chamber 2, and secured on the ends of the lower surface of the liquid chamber 2. The discharging hole 3a, which is a through hole, is formed in approximately the center of the membrane 3, and the cell ink A contained in the liquid chamber 2 is discharged through the discharging hole 3a in the form of a liquid droplet D in response to deformation of the membrane 3.

The membrane 3 is deformed by the piezoelectric element 4.

The shape of the membrane 3 when seen in a plan view perspective is not particularly limited and may be appropriately selected depending on the intended purpose. Examples of the shape of the membrane 3 include a circular shape, an elliptic shape, and a quadrangular shape. A shape matching the shape of the bottom of the liquid chamber 2 is preferable.

The material of the membrane 3 is not particularly limited and may be appropriately selected depending on the intended purpose. Examples of the material of the membrane 3 include metallic materials, ceramic materials, and polymeric materials. A material having a certain degree of hardness is preferable. When the material of the membrane 3 has a certain degree of hardness, the membrane 3 does not easily undergo vibration, and vibration of the membrane 3 can be easily suppressed.

Examples of the metallic materials include stainless steel, nickel, and aluminum.

Examples of the ceramic materials include silicon dioxide, alumina, and zirconia.

In the present embodiment, the discharging hole 3a is formed in approximately the center of the membrane 3 in substantially a perfect circle shape.

The shape of the discharging hole 3a is not particularly limited and may be appropriately selected depending on the intended purpose.

Examples of the shape of the discharging hole 3a include a perfect circle shape.

When the shape of the discharging hole 3a is a perfect circle shape, the diameter of the discharging hole 3a is not particularly limited, may be appropriately selected depending on the intended purpose, and is preferably 20 micrometers or greater but 200 micrometers or less. The diameter of the discharging hole 3a in the preferable range is advantageous in terms of stabilization of the shape of the liquid droplets to be discharged.

The piezoelectric element 4 is disposed at the lower surface side of the membrane 3.

The shape of the piezoelectric element 4 is preferably a shape matching the shape of the membrane 3. For example, when the shape of the membrane 3 when seen in the plan view perspective is a circular shape, it is preferable to form the piezoelectric element 4 having an annular (ring-like) planar shape around the discharging hole 3a.

The piezoelectric element 4 has a structure obtained by providing the upper surface and the lower surface of a piezoelectric material with electrodes across which a voltage is to be applied. When a voltage is applied across the upper and lower electrodes of the piezoelectric element 4, a compressive stress is applied in the horizontal direction of the drawing sheet, making it possible for the membrane 3 to deform or vibrate.

Examples of the piezoelectric material include lead zirconate titanate, bismuth iron oxide, metal niobate, and barium titanate, and materials obtained by adding metals or different oxides to these materials.

In the present embodiment, the piezoelectric element 4 is configured to deform the membrane 3. However, this is non-limiting, and any other mode may be employed. In any other mode, for example, a material having a different coefficient of linear expansion from the coefficient of linear expansion of the membrane 3 may be pasted over the membrane 3, and may be heated to deform the membrane 3 utilizing the difference between the coefficients of linear expansion. In this mode, it is preferable to dispose a heater near the material having the different coefficient of linear expansion, and cause the membrane 3 to deform or vibrate in accordance with ON or OFF of the heater.

The driving unit 5 can output a discharging signal Pj to the piezoelectric element 4 as a driving signal. By outputting the discharging signal Pj to the piezoelectric element 4, the driving unit 5 can cause the membrane 3 to deform and discharge the cell ink A contained in the liquid chamber 2 in the form of a liquid droplet D. Further, by causing the membrane 3 to deform by means of the discharging signal Pj set to a predetermined period, the driving unit 5 can cause the liquid to be discharged under resonant vibration of the membrane 3.

The driving unit 5 can output a suppressing signal Ps to the piezoelectric element 4 as a driving signal. By outputting the suppressing signal Ps to the piezoelectric element 4 after a liquid droplet D is discharged, the driving unit 5 can suppress residual vibration of the membrane 3. Therefore, the liquid droplet forming device 1 can suppress the residual vibration of the membrane 3 quickly without waiting for the residual vibration to decay naturally, and can hence increase the number of times of discharging per unit time. Furthermore, the liquid droplet forming device 1 can perform more minute control of the liquid droplet amount, because the liquid droplet forming device 1 can reduce occurrence of troubles due to the residual vibration such as a satellite formed when a liquid droplet is split or a mist formed when a liquid droplet scatters minutely.

### [Liquid Droplet Forming Process (Operation) of Liquid Droplet Forming Device]

A process through which the liquid droplet forming device forms a liquid droplet will be described.

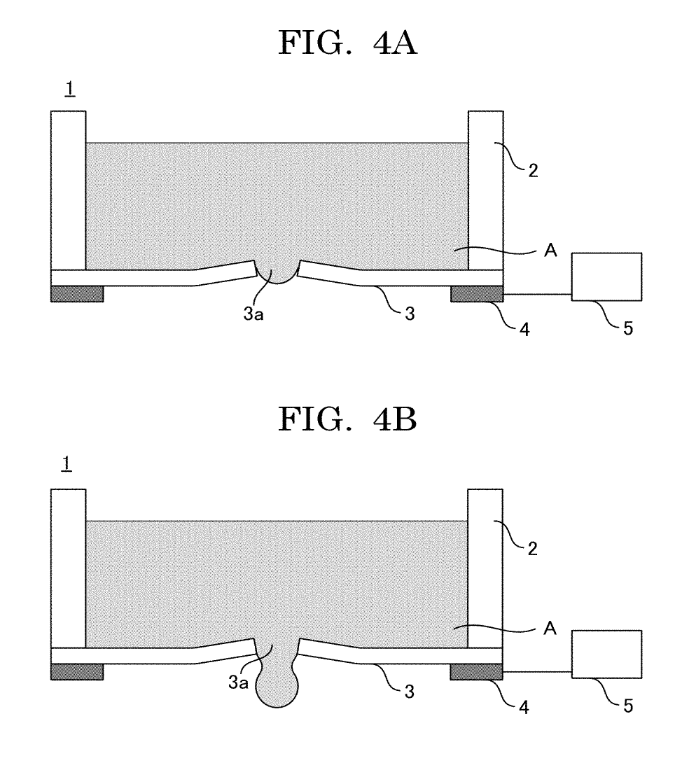

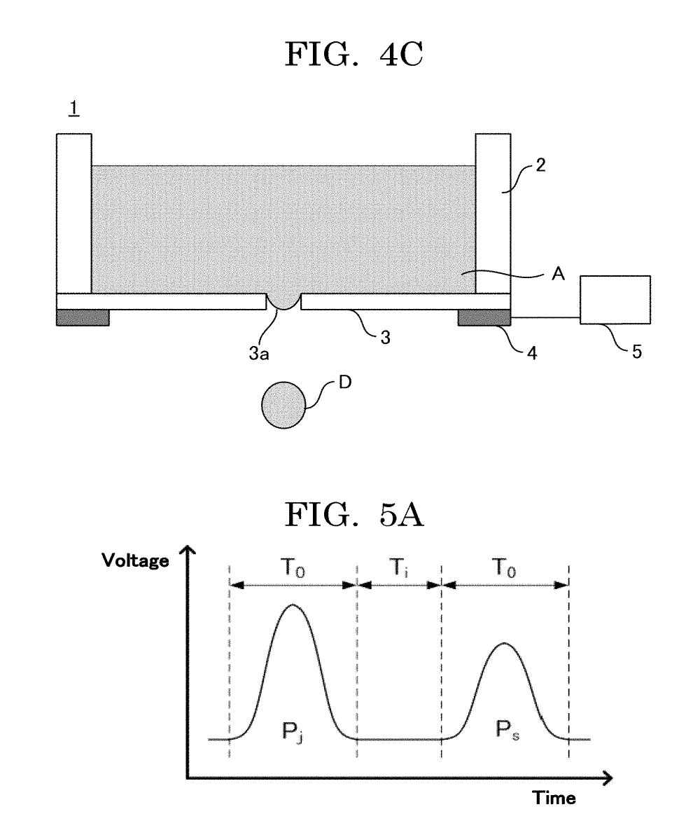

FIG. 3 is a graph plotting an example of the discharging signal and the suppressing signal. FIG. 4A to FIG. 4C are views illustrating an example of an operation of the liquid droplet discharging device.

When the discharging signal Pj and the suppressing signal Ps plotted in FIG. 3 are output to the piezoelectric element 4, a liquid droplet D can be formed and residual vibration of the membrane 3 can be suppressed as well, as illustrated in FIG. 4A to FIG. 4C.

First, when the discharging signal Pj is output as plotted in FIG. 3, the membrane 3 rapidly deforms as illustrated in FIG. 4A to push out the cell ink A contained in the liquid chamber 2 downwards through the discharging hole 3a.

The discharging signal Pⱼ is not particularly limited and may be appropriately selected depending on the intended purpose. As the discharging signal Pⱼ, a signal based on the natural vibration period T₀ of the membrane 3 is preferable in terms of discharging the cell ink A at a lower voltage by means of the membrane 3. In the present embodiment, by setting the time for which the discharging signal Pⱼ is output, i.e., the time for which the applied voltage is raised, to T₀/2, it is possible to discharge the cell ink A at a lower voltage by means of the membrane 3.

The natural vibration period T₀ of the membrane 3 can be measured with, for example, a laser Doppler vibrometer (LV-1800, available from Ono Sokki Co., Ltd.).

Next, as plotted in FIG. 3, during a time for which a constant voltage is applied to the piezoelectric element 4, i.e., during the interval time Tᵢ from when outputting of the discharging signal Pⱼ is ended until when outputting of the suppressing signal Pₛ is started, a liquid droplet D from the discharging hole 3a grows as illustrated in FIG. 4B. During this interval time Tᵢ, the vibration of the membrane 3 due to deformation during discharging is remaining.

Then, when the suppressing signal Pₛ is output as plotted in FIG. 3, the liquid droplet D is formed when the membrane 3 returns to the original state as illustrated in FIG. 4C and the residual vibration of the membrane 3 is suppressed as well.

The suppressing signal Pₛ is not particularly limited and may be appropriately selected depending on the intended purpose, and a signal based on the natural vibration period T₀ of the membrane 3 is preferable. When the suppressing signal Pₛ is a signal based on the natural vibration period T₀ of the membrane 3, it is possible to suppress the residual vibration of the membrane 3 with a low energy and in a short time.

The voltage of the suppressing signal Pₛ is set to lower than or equal to the highest voltage of the discharging signal Pⱼ. When the voltage of the suppressing signal Pₛ is higher than the highest voltage of the discharging signal Pⱼ, the suppressing signal Pₛ may generate unneeded vibration and tend to invite long persistence of the residual vibration.

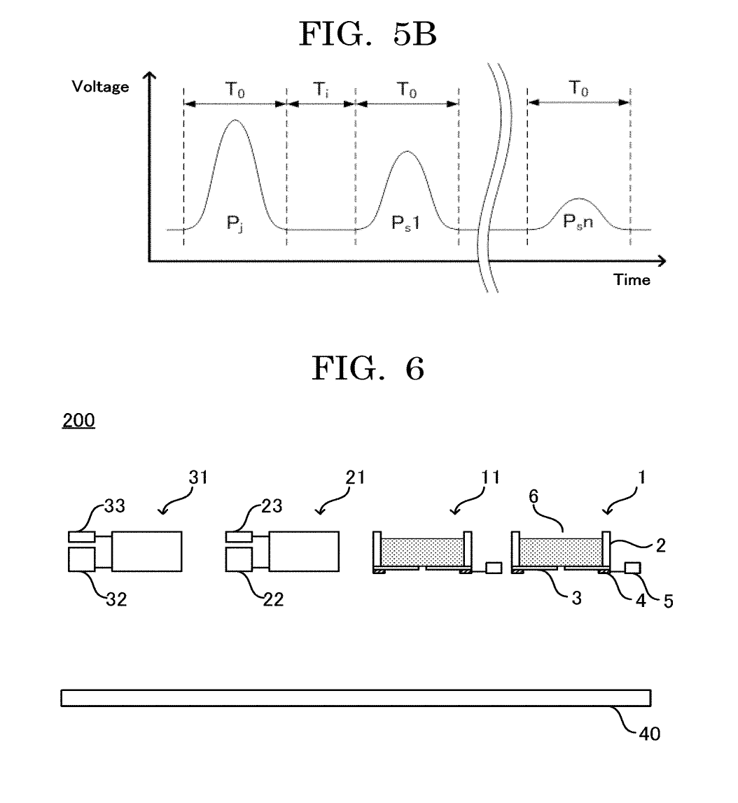

In the foregoing description, the voltage signal at the rise of the pulsed driving signal plotted in FIG. 3 is the discharging signal, and the voltage signal at the fall is the suppressing signal. However, this is non-limiting. For example, the discharging signal and the suppressing signal may be as plotted in FIG. 5A and FIG. 5B.

As plotted in FIG. 5A, the discharging signal Pⱼ may be, for example, a triangle wave, a sine wave, a rectangular wave, and a triangle wave passed through a low pass filter to have gentle edges. In this case, it is preferable to match the period of, for example, a triangle wave with the natural vibration period T₀ of the membrane 3.

The suppressing signal Pₛ is not particularly limited and may be appropriately selected depending on the intended purpose so long as the suppressing signal Pₛ is a signal based on the natural vibration period T₀ of the membrane 3. The suppressing signal Pₛ may be, for example, a triangle wave, a sine wave, a rectangular wave, and a triangle wave passed through a low pass filter to have gentle edges. In this case, the period of, for example, a triangle wave is matched with the natural vibration period T₀ of the membrane 3.

When it is impossible to suppress the residual vibration of the membrane 3 by outputting the suppressing signal Pₛ only once, the liquid droplet forming device 1 may output a plurality of suppressing signals Pₛ as plotted in FIG. 5B. Also in this case, the period of, for example, a triangle wave is matched with the natural vibration period T₀ of the membrane 3.

[Cell Contained Member Producing Apparatus Mounded with Liquid Droplet Forming Device]

FIG. 6 is a schematic view illustrating an example of a cell contained member producing apparatus mounted with a plurality of liquid droplet forming devices.

As illustrated in FIG. 6, a cell contained member producing apparatus 200 includes at least a stage section 40 over which a base material is secured, a liquid droplet forming device 1 and a liquid droplet forming device 11 that are configured to discharge a cell ink, a coating liquid discharging head 21, and a cell adhesive material discharging head 31.

The coating liquid discharging head 21 includes a driving unit 22 and a liquid chamber 23 configured to retain a coating liquid. The cell adhesive material discharging head 31 includes a driving unit 32 and a liquid chamber 33 configured to retain a cell adhesive material ink.

The number of each device and head may be changed as needed.

As the cell adhesive material discharging head 31, the liquid droplet forming device 1 may be used or any other inkjet head may be used.

Examples of the any other inkjet head include an industrial inkjet head (MH2420). The apparatus may also include a holding member configured to hold the inkjet heads, and a mechanical section configured to control the relative positions of the stage and the inkjet heads.

(Description about Method for Producing Tissue)

### —Preparation of Cell Adhesive Material Ink—

A cell adhesive material was dissolved in, for example, pure water, to prepare a cell adhesive material ink B. Here, the content of the cell adhesive material is preferably from 0.001% through 5% and more preferably from 0.005% through 1%. When the content of the cell adhesive material is in the preferable range, there are advantages that expression of the cell adhesive function is smooth, and that discharging of the cell adhesive material ink by an inkjet method is smooth.

As needed, other additive materials such as a humectant, a dispersant, and a pH adjustor may be added. It is more preferable that the boiling point is about from room temperature through 90 degrees C.

—Discharging of Cell Adhesive Material Ink into Holes—

In order to improve adhesiveness of cells with a base material 130, the cell adhesive material was located over the surface of the base material 130. Examples of the method for locating the cell adhesive material include a method of seeding the cell adhesive material over the entire surface, and a method of discharging the cell adhesive material into holes 120 using the producing apparatus 200 of FIG. 6. Of these methods, the method using the producing apparatus 200 of FIG. 6 is preferable because accurate discharging to desired positions is possible.

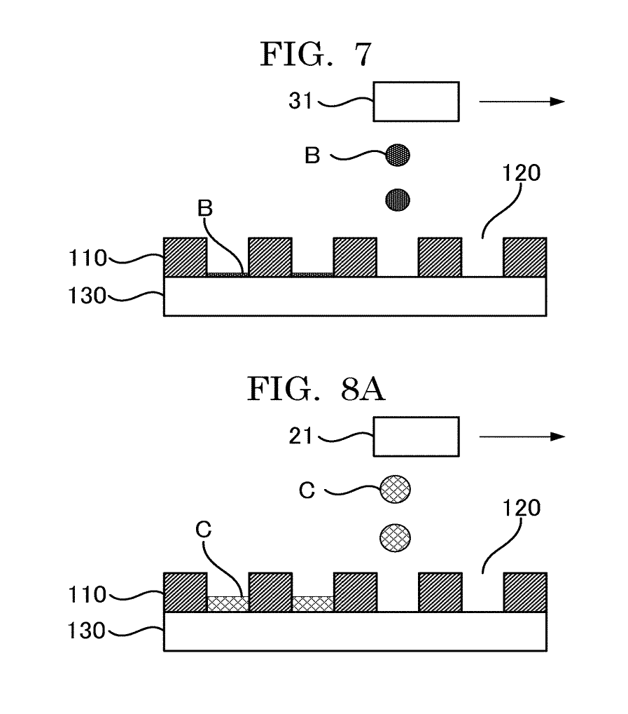

FIG. 7 is a view illustrating an example of an operation for discharging the cell adhesive material ink.

As illustrated in FIG. 7, by discharging the cell adhesive material ink B into the holes 120 using the cell adhesive material discharging head 31, it is possible to control the cell adhesive material to a fixed quantity in conformation with the pattern. Here, the driving signal is not particularly limited so long as the cell adhesive material ink B can be discharged through the nozzle. The cell adhesive material may be located before the structural layer is laminated over the base material.

—Injection of Coating Liquid into Holes—

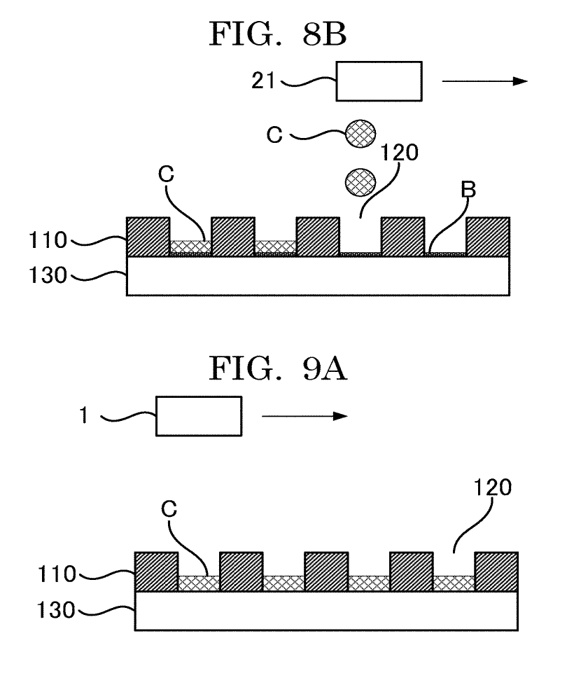

FIG. 8A is a view illustrating an example of an operation for discharging the coating liquid.

As illustrated in FIG. 8A, by discharging a coating liquid C into the holes 120 bottomed by the base material 130 using the coating liquid discharging head 21, it is possible to control the coating liquid C to a fixed quantity in conformation with the pattern. The driving signal is not particularly limited so long as the coating liquid C can be discharged through the nozzle. As illustrated in FIG. 8B, it is preferable to discharge the cell adhesive material ink B before discharging the coating liquid C.

In this way, before a cell ink A is discharged from the cell discharging head 1, the coating liquid C is previously injected into the holes 120 for suppression of drying of the cells and suppression of impacts on the cells when landing on the substrate.

—Injection of Cell Ink into Holes—

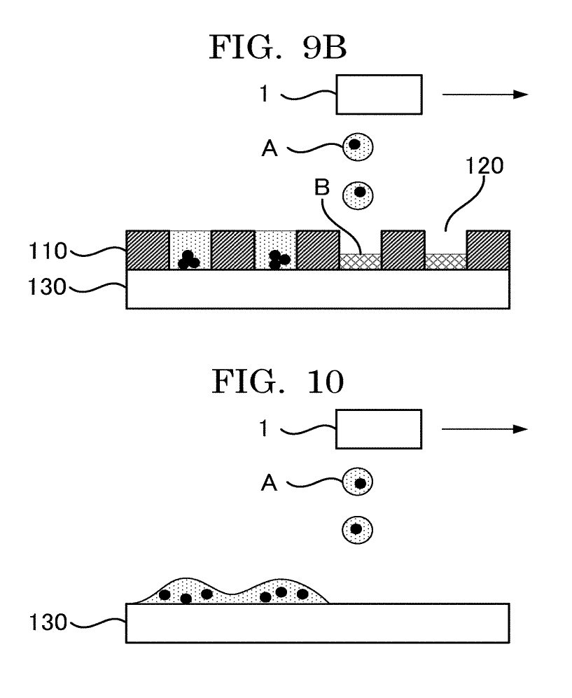

FIG. 9A and FIG. 9B are views illustrating an example of an operation for discharging the cell ink.

As illustrated in FIG. 9A and FIG. 9B, by discharging the cell ink A into the holes 120 bottomed by the base material 130 using the cell discharging head 1, it is possible to control the cell ink A to a fixed quantity in conformation with the pattern.

A relationship between the liquid depth of the coating liquid C and the cell survival rate when the liquid droplet discharging speed at which the cell ink A was discharged from the cell discharging head 1 was set to 0.1 m/s, 1.0 m/s, and 10 m/s was evaluated according to the criteria described below. The results are presented in Table 1.

A: The average of the cell survival rate per hole was 75% or higher.

B: The average of the cell survival rate per hole was lower than 75%.

TABLE 1

| | | Liquid thickness of coating liquid (micrometer) | | |
|---|---|---|---|---|
| | | 0 | | |
| | | (no coating liquid) | 5 | 100 |
| Cell ink | 0.1 | B | A | A |
| discharging | 1.0 | B | A | A |
| speed (m/s) | 10.0 | B | A | A |

From the results of Table 1, it was confirmed that regardless the discharging speed in the discharging speed range of from 0.1 m/s through 10 m/s, injecting the coating liquid C into the holes 120 even with a liquid thickness of about 5 micrometers significantly improved the cell survival rate as compared with when there was no coating liquid C.

Providing the coaling liquid C in a high liquid amount is effective when taking into consideration suppression of drying of the cells and suppression of impacts on the cells when landing, but this relatively reduces the cell ink A that can be contained in the holes 120 and reduces the cell density. Therefore, the liquid amount of the coating liquid C may be adjusted depending on the shape of a desired tissue.

The cell ink A is discharged into the holes 120 from the cell discharging head 1. Coalescing of adjacent liquid droplets is suppressed by the portioning wall formed of the structural layer 110. Furthermore, because it is possible to increase the number of liquid droplets to the same position, it is also possible to increase the cell density. Further, because the coating liquid C is injected in the holes 120, impacts on the cells when landing on the base material 130 can be suppressed. Moreover, with the coating liquid C, which is a solution containing components needed for formation and sustainment of a tissue, preventing drying, and adjusting the external environment such as the osmotic pressure, it is possible to improve the cell survival rate and cope with enlargement and complication of a tissue.

By repeating the steps described above, it is possible to build a three-dimensional, complicated tissue. Whether or not to peel or keep the structural layer 110 in each layer structure is not limited because this depends on the purpose for which a tissue is built and biocompatibility of the structural layer.

FIG. 10 is a view illustrating an example of an operation for discharging a cell ink by an existing inkjet method.

As illustrated in FIG. 10, because cells are dispersed in the cell ink, as the cell resolution and the cell density are higher, liquid droplets landed over the base material 130 are more likely to coalesce with each other if adjacent liquid droplets are close, leading to a failure to achieve the intended cell positions and the intended cell number. Hence, if a plurality of kinds of cells coalesce with each other, the quality of the tissue becomes poor. Moreover, because liquid droplets of the cell ink discharged from the cell discharging head 1 directly impinge on the base material 130, the cells are highly probably damaged. Furthermore, after discharged, the cell ink A is exposed to the atmosphere and starts to dry.

Hence, it may be feared that due to enlargement and complication of a tissue, cells previously discharged may dry and the cell survival rate may become poor.

### EXAMPLES

The present disclosure will be described below by way of Examples. The present disclosure should not be construed as being limited to these Examples.

### Example 1

### <From Formation of Holes in Structural Layer Till Formation of Cell Adhesive Material>

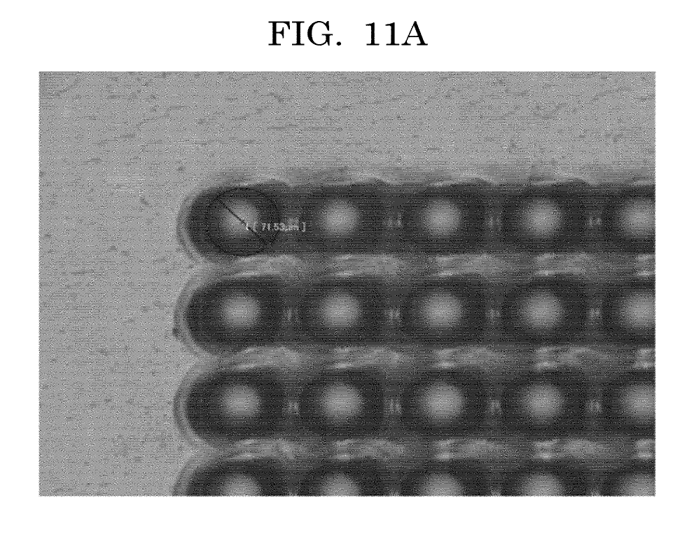

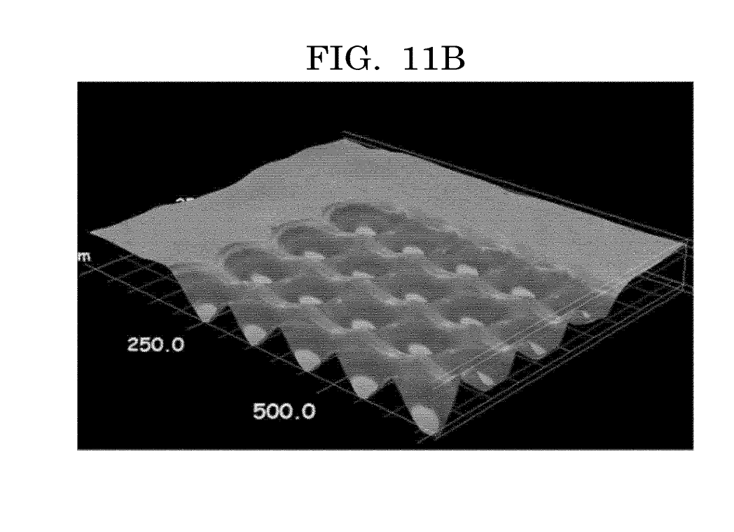

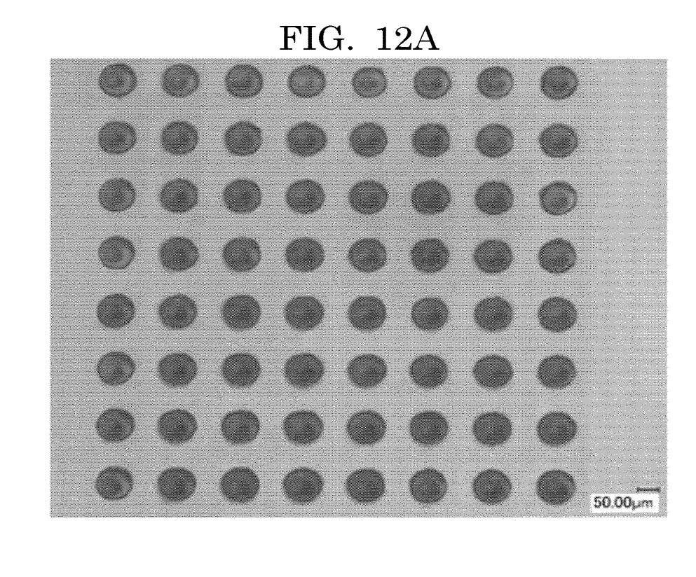

Four masking tapes having a thickness of 70 micrometers (N-380, available from Nitto Denko Corporation) were pasted in an overlapping state (with a thickness of 280 micrometers) over a glass slide, and holes with a diameter ϕ of 100 micrometers as illustrated in FIG. 11A and FIG. 11B were subsequently formed at a pitch of 150 micrometers with a laser process machine (SPECTRA-PHYSICS H10-106QW J80-8SS42 HIPPO), to form a structural layer. Further, for positional alignment with an evaluation substrate serving as a base material, alignment marks having a diameter ϕ of 100 micrometers were also formed.

Next, the overlapping four masking tapes were peeled from the glass slide, and positional alignment was subsequently performed with alignment marks on the evaluation substrate and the alignment marks on the masking tapes, to form a desired evaluation substrate.

Then, a cell adhesive material solution was prepared using matrigel (CORNING MATRIGEL BASEMENT MEMBRANE MATRIX) as a cell adhesive material at 0.1% by weight. The cell adhesive material solution was filled in the liquid chamber of the cell adhesive material discharging head, and liquid droplets were discharged in an amount of 900 pL (300 pL×3 droplets) into each hole through the nozzle and dried, to form a cell adhesive material.

### <Preparation of Cell Ink>

A green fluorescent dye (product name: CELL TRACKER GREEN, available from Life Technologies Corporation) was dissolved in dimethyl sulfoxide at a concentration of 10 mmol/L (mM), and mixed with a serum-free Dulbecco's modified Eagle's medium (available from Life Technologies Corporation), to prepare a fluorescent dye-containing serum-free medium having a concentration of 10 micromoles/L (micromol).

Next, the fluorescent dye-containing serum-free medium (5 mL) was added in a dish of cultured NIH/3T3 cells (CLONE 5611, JCRB Cell Bank) and the resultant was cultured in an incubator (KM-CC17RU2, available from Panasonic Corporation, in a 5% by volume CO₂ environment at 37 degrees C.) for 30 minutes. Subsequently, the supernatant was removed using an aspirator. A phosphate buffered saline (available from Life Technologies Corporation, hereinafter may also be referred to as “PBS (−)”) (5 mL) was added in the dish and then aspirated with an aspirator, to wash the surface. The washing operation with PBS (−) was repeated twice, and then a 0.05% by mass trypsin-0.05% by mass EDTA solution (available from Life Technologies Corporation) was added in an amount of 2 mL per dish. Next, the resultant was heated in an incubator for 5 minutes, and after the cells were stripped from the dish, D-MEM (4 mL) containing a 10% by mass fetal bovine serum (hereinafter, may also referred to as “FBS”) and a 1% by mass antibiotic (ANTIBIOTIC-ANTIMYCOTIC MIXED STOCK SOLUTION (100×), available from Nacalai Tesque Inc.) was added in the dish. Next, the cell ink in which trypsin was deactivated was removed into a 50 mL centrifuge tube for centrifugation (product name: H-19FM, available from KOKUSAN Co., Ltd., at 1,200 rpm, for 5 minutes, at 5 degrees C.), and then the supernatant was removed with an aspirator. After the removal, the D-MEM (2 mL) containing the 10% by mass FBS and the 1% by mass antibiotic was added in the centrifuge tube, followed by gentle pipetting, to obtain a cell ink in which the cells were dispersed.

The cell ink (10 microliters) was taken out into an Eppendorf tube, into which a culture medium (70 microliters) was added. Subsequently, the resultant (10 microliters) was taken out into another Eppendorf tube, into which a 0.4% by mass Trypan blue stain (10 microliters) was added, followed by pipetting. The stained cell ink (10 microliters) was taken out and poured onto a plastic slide formed of PMMA. Using a product named: COUNTESS AUTOMATED CELL COUNTER (available from Invitrogen), the number of cells was counted to obtain the number of cells, to obtain a cell ink for which the number of cells was counted. PBS (−) was used as a dispersion medium. Glycerin (molecular biology grade, available from Wako Pure Chemical Industries Ltd.) serving as a humectant was dissolved in the PBS (−) at a mass ratio of 0.5% by mass, and the NIH/3T3 cell ink was dispersed in the dispersion medium at 6×10⁶ cells/mL, to obtain a cell ink.

<From Injection of Coating Liquid into Holes Till Formation of Cell Pattern>

Next, a culture medium (D-MEM containing 10% by mass FBS and 1% by mass antibiotic) serving as a coating liquid was filled in the liquid chamber of the coating liquid discharging head, and a liquid droplet was discharged through the nozzle in an amount of 300 pL (300 pL×1 droplet) (with a liquid depth of 40 micrometers) into each hole of the evaluation substrate over which the cell adhesive material was formed.

Subsequently, the prepared cell ink was filled in the liquid chamber of the cell discharging head, and liquid droplets were discharged through the nozzle in an amount of 1,500 pL (300 pL×5 droplets) into each hole at a liquid droplet speed of 1.0 m/s, intending to locate ten cells.

After the last cell ink had landed, the evaluation substrate was left to stand still for 10 minutes and then subjected to culturing in an incubator (KM-CC17RU2, available from Panasonic Corporation, in a 5% by volume CO₂ environment at 37 degrees C.) for 24 hours.

The obtained cell pattern was evaluated in the manners described below.

### [Cell Pattern Shape Accuracy (Evaluation of Pattern Resolution)]

The cell pattern shape accuracy was evaluated according to the criteria described below, using a fluorescence microscope (CKX41, available from Olympus Corporation).

A: The pitch of the cell adhesive pattern was 300 micrometers or less.

B: The pitch of the cell adhesive pattern was greater than 300 micrometers.

[Probability at which Predetermined Number of Cells can be Located at Predetermined Cell Adhesive Portions (Evaluation of Cell Number Control)]

The number of cells contained per hole having a diameter (ϕ) of 100 micrometers was calculated based on a fluorescence image, and evaluated according to the criteria described below. The evaluation targets were all holes into which liquid droplets were discharged, intending to locate ten cells.

A: The number of cells was within ±20% of the intended number at more than or equal to 80% of the holes.

B: The number of cells was within ±20% of the intended number at less than 80% of the holes.

In the present Example, the intended number was the reference, but the average value of the number of cells may be used as the reference.

[Probability at which Number of Cells Located was Desired Number (Evaluation of Cell Density)]

The number of cells contained per hole having a diameter (ϕ) of 100 micrometers was calculated based on a fluorescence image, and evaluated according to the criteria described below. The evaluation targets were twenty holes into which liquid droplets were discharged, intending to locate ten cells.

A: The number of cells was eight or more at more than or equal to 80% of the holes

B: The number of cells was eight or more at less than 80% of the holes.

[Survival Rate of Landed Cells after Predetermined Time had Passed (Evaluation of Survival Rate of Cells)]

A stock solution obtained by dissolving propidium iodide as a dead cell nuclear stain (CELLSTAIN-PI, available from Dojindo Laboratories) in distilled water at 1 mg/mL was added at 10 microliters/5 mL onto the cell pattern having been left to stand still for 10 minutes. Subsequently, the resultant was cultured in an incubator for 10 minutes. Then, the ratio of dead cells present per hole was calculated using a fluorescence microscope and evaluated according to the criteria described below. The evaluation targets were twenty holes into which liquid droplets were discharged, intending to locate ten cells.

A: The average survival rate of cells per hole was 75% or higher.

B: The average survival rate of cells per hole was lower than 75%.

### Example 2

The same evaluations as in Example 1 were performed except that unlike in Example 1, the structural layer was changed from the masking tapes to PDMS. The results are presented in Table 2.

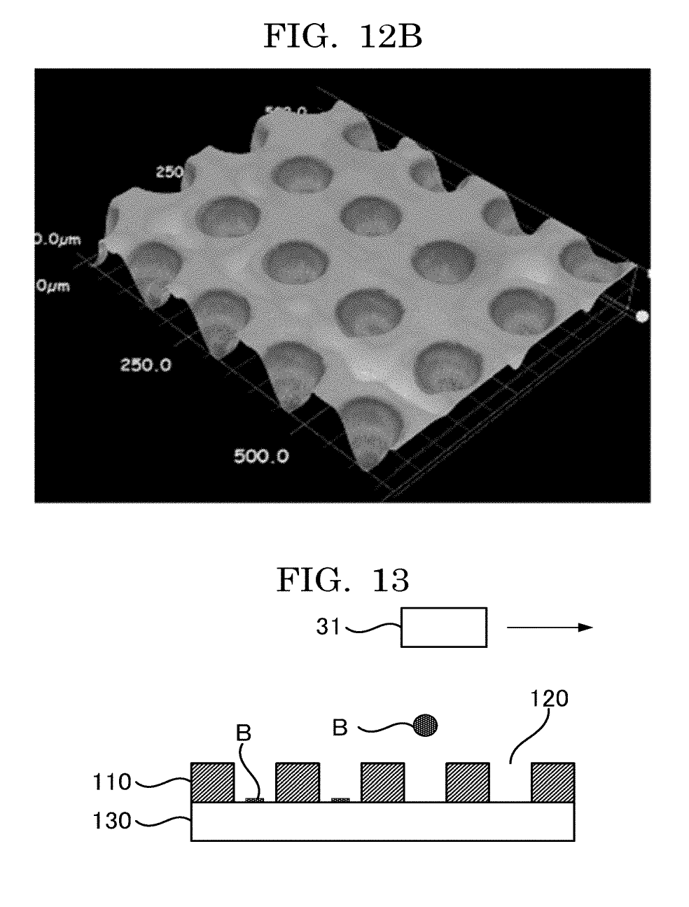

As a method for forming a bulk of PDMS, PDMS (available from Dow Corning Toray Co., Ltd., SYLGARD184), which was a deaerated mixture of a main agent and a curing agent at 10:1, was coated over a glass slide (available from Matsunami Glass Ind., Ltd., MICRO SLIDE GLASS S1112) and thermally cured at 70 degrees C. for 2 hours. Then, by laser machining, holes as illustrated in FIG. 12A and FIG. 12B were formed in the structural layer formed of PDMS.

### Example 3

The same evaluations as in Example 2 were performed except that unlike in Example 2, the cell adhesive material was changed to collagen at a mass ratio of 0.05% by mass (CELLMATRIX TYPE 1-A, available from Nitta Gelatin Inc.). The results are presented in Table 2.

### Example 4

The same evaluations as in Example 2 were performed except that unlike in Example 2, the cell adhesive material was changed to fibrin. The fibrin was obtained by mixing fibrinogen (Fibrinogen from bovine plasma F8630, available from SIGMA-ALDRICH Co., LLC.) (mass ratio of 0.2% by mass) with thrombin (Thrombin from bovine plasma T4648, available from SIGMA-ALDRICH Co., LLC.) (mass ratio of 0.2% by mass), and leaving the mixture to stand still at room temperature overnight. The results are presented in Table 2.

### Example 5

The same evaluations as in Example 2 were performed except that unlike in Example 2, the cell adhesive material was changed to RGD peptide (for biochemical use, available from Wako Pure Chemical Industries Ltd.). The results are presented in Table 2.

### Example 6

The same evaluations as in Example 2 were performed except that unlike in Example 2, the cell adhesive material was changed to gelatin particles (APH-250, available from Nitta Gelatin Inc.) (mass ratio of 2% by mass). The results are presented in Table 2.

### Example 7

The same evaluations as in Example 2 were performed except that unlike in Example 2, the number of liquid droplets of the cell adhesive material in each hole was changed to one liquid droplet. The results are presented in Table 2.

FIG. 13 is a view illustrating an example of an operation for discharging the cell adhesive material ink in Example 7.

As illustrated in FIG. 13, adjusting the amount of the cell adhesive material to land over the substrate enables suppression of formation of the cell adhesive material over the side surfaces of the holes serving as the partitioning walls. This makes it possible for all discharged cells to be formed over the substrate, making control on the number of cells to be formed over the substrate highly accurate.

Moreover, during observation of the cells formed over the substrate with a microscope, it is possible to reduce a margin of error of measurement on the partitioning walls susceptible to variation due to the meniscus of the liquid.

### Example 8

The same evaluations as in Example 2 were performed except that unlike in Example 2, the holes were formed to have a shape in which the area of the opening in a surface at the substrate side was smaller than the area of the opening at the opposite side. The results are presented in Table 2.

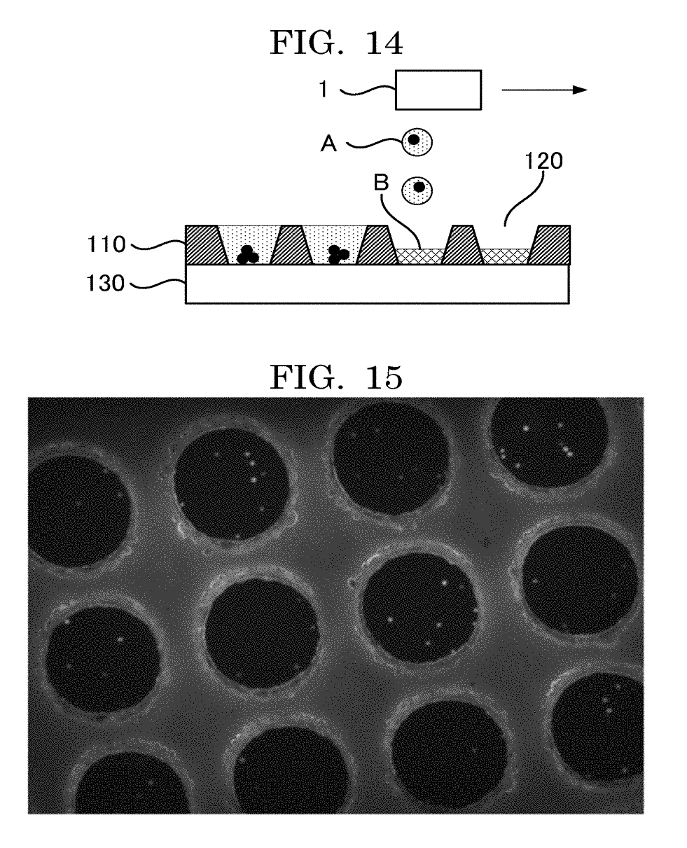

FIG. 14 is a view illustrating an operation for discharging a cell ink in Example 8.

As illustrated in FIG. 14, the hole area at the liquid droplet discharging side can be broadened with the cell resolution and the cell density maintained, making it possible to suppress variation in the number of cells due to fluctuation of the landing position due to the cell discharging head.

### Example 9

The same evaluations as in Example 2 were performed except that unlike in Example 2, a cell ink 1 in which NIH/3T3 was dispersed and a cell ink 2 in which normal human dermal fibroblast (CC-2509, Lonza, hereinafter referred to as “NHDF”) was dispersed were prepared and alternately located in the holes of the structural layer, to perform evaluation on a plurality of kinds of cells.

The cell ink 2 was prepared in the same manner as the cell ink 1, except that the cell was changed to NHDF and the dye was changed to a red fluorescent dye (CELL TRACKER RED, available from Life Technologies Corporation). The results are presented in Table 2. FIG. 15 is an image of a cell ink located in a state of being dyed into two kinds.

Because a plurality of kinds of cells were used in Example 9, the pattern accuracy in a state in which the cells adhered was evaluated according to the criteria described below, using a fluorescence microscope (CKX41, available from Olympus Corporation).

A: The pitch of the alternately located cell pattern was 300 micrometers or less.

B: The pitch of the alternately located cell pattern was greater than 300 micrometers.

### Comparative Example 1

The same evaluations as in Example 9 were performed except that unlike in Example 9, no structural layer was disposed over the substrate. The results are presented in Table 2.

Because there were no holes formed in a structural layer in Comparative Example 1, adjacent liquid droplets coalesced with each other, making it impossible to make cells adhere to intended adhesion positions. Hence, the pattern could not be formed at the intended cell resolution and the intended cell density. Moreover, because it was impossible to have the coating liquid contained, the cell survival rate could not be achieved due to impinging on the substrate.

### Comparative Example 2

The same evaluations as in Example 9 were performed except that unlike in Example 9, no coating liquid was formed. The results are presented in Table 2.

Because there were holes formed in the structural layer in Comparative Example 2, patterning of a plurality of kinds of cells was achieved in addition to the cell resolution, the cell number control, and the cell density. However, because there was no coating liquid for preventing drying of the cells and suppressing impacts, the cell survival rate standard could not be achieved.

### Comparative Example 3

The same evaluations as in Example 9 were performed except that unlike in Example 9, the cell inks of NIH/3T3 and NHDF were prepared, and instead of employing an inkjet method for locating cells, the cell inks were seeded manually by 5×10⁴ cells/mL per cell ink and 1×10⁵ cells/mL in total. The results are presented in Table 2.

In Comparative Example 3, the positional accuracy in seeding was poor because cells were not located by an inkjet method. Therefore, cells could not be located accurately in the intended holes, but cells were located in adjacent holes or outside the holes such as over the structural layer and interfered and coalesced with liquid droplets in adjacent holes, resulting in failure to achieve the cell number control standard. In addition, the standard of the pattern formed of the plurality of kinds of cells could not be achieved, because NIH/3T3 and NHDF coalesced with each other.

In Comparative Example 3, the “member for containing cells”, which was in the state before cells were seeded, corresponds to Example.

TABLE 2

| | Ex. 1 | Ex. 2 | Ex. 3 | Ex. 4 | Ex. 5 | Ex. 6 | Ex. 7 |
|---|---|---|---|---|---|---|---|
| Method for forming cell pattern | IJ | IJ | IJ | IJ | IJ | IJ | IJ |
| Method for forming cell adhesive | IJ | IJ | IJ | IJ | IJ | IJ | IJ |
| material pattern | | | | | | | |
| Method for forming coating liquid | IJ | IJ | IJ | IJ | IJ | IJ | IJ |
| pattern | | | | | | | |
| Material of structural layer | Masking | PDMS | PDMS | Masking | PDMS | PDMS | PDMS |
| | tape | | | tape | | | |
| Coating liquid | Culture | Culture | Culture | Culture | Culture | Culture | Culture |
| | medium | medium | medium | medium | medium | medium | medium |
| Cell adhesive material | Matrigel | Matrigel | Collagen | Fibrin | RGD | Gelatin | Matrigel |
| | | | | | peptide | particles | |
| Number of liquid droplets of cell | 5 | 5 | 5 | 5 | 5 | 5 | 1 |
| adhesive material | | | | | | | |
| Opening shape of holes | ST | ST | ST | ST | ST | ST | ST |
| Kind of cell | 1 | 1 | 1 | 1 | 1 | 1 | 1 |
| Cell resolution | A | A | A | A | A | A | A |
| Cell number control | A | A | A | A | A | A | A |
| Cell density | A | A | A | A | A | A | A |
| Cell survival rate | A | A | A | A | A | A | A |
| Plurality of cells | — | — | — | — | — | — | — |

| | | | Comp. | Comp. | Comp. |
|---|---|---|---|---|---|
| | Ex. 8 | Ex. 9 | Ex. 1 | Ex. 2 | Ex. 3 |
| Method for forming cell pattern | IJ | IJ | IJ | IJ | Manually |
| Method for forming cell adhesive | IJ | IJ | IJ | IJ | IJ |
| material pattern | | | | | |
| Method for forming coating liquid | IJ | IJ | IJ | IJ | IJ |
| pattern | | | | | |
| Material of structural layer | PDMS | PDMS | Absent | PDMS | PDMS |
| Coating liquid | Culture | Culture | Absent | Absent | Culture |
| | medium | medium | | | medium |
| Cell adhesive material | Matrigel | Matrigel | Matrigel | Matrigel | Matrigel |
| Number of liquid droplets of cell | 5 | 5 | 5 | 5 | 5 |
| adhesive material | | | | | |
| Opening shape of holes | Taper | ST | ST | ST | ST |
| Kind of cell | 1 | 2 | 2 | 2 | 2 |
| Cell resolution | A | A | B | A | A |
| Cell number control | A | A | A | A | B |
| Cell density | A | A | B | A | A |
| Cell survival rate | A | A | B | B | A |
| Plurality of cells | — | A | B | A | B |

As described above, with a structural layer including a plurality of holes over a base material, the member for containing cells of the present disclosure can contain cells dividedly in the plurality of holes when a cell suspension is discharged into the holes, making it possible to suppress mislocation of cells due to coalescing of landed liquid droplets.

Furthermore, by containing the landed cell suspension in the holes, the member for containing cells of the present disclosure can reduce the surface area of the cell suspension contacting the atmosphere, making it possible to suppress drying of the cells.

Moreover, by including the coating liquid coating the plane of the base material exposed through the holes in the holes, the member for containing cells of the present disclosure can suppress cells discharged by an inkjet method from being damaged when landing, and can suppress drying of the cells.

Aspects of the present disclosure are as follows, for example.

<1> A method for producing a cell locating plate, the method including:
laminating a structural layer over a plane of a base material, the structural layer being a layer that is formed of a cell non-adhesive material and in which a plurality of holes are formed;
coating a coating liquid in the holes and over the plane of the base material exposed through the holes; and
discharging a cell suspension onto the plane coated with the coating liquid.
<2> The method for producing a cell locating plate according to <1>, the method further including
attaching a cell adhesive material in the holes.
<3> The method for producing a cell locating plate according to <2>, wherein the cell adhesive material is contactless with side surfaces of the holes.
<4> The method for producing a cell locating plate according to <1>, wherein the plane of the base material exposed through the holes in the structural layer is a surface onto which liquid droplets of the cell suspension discharged by an inkjet method land.
<5> The method for producing a cell locating plate according to <1>, wherein the cell non-adhesive material is a water-repellent material or a silicon-containing material.
<6> The method for producing a cell locating plate according to <1>, wherein a minimum thickness of the coating liquid is 5 micrometers or greater.
<7> The method for producing a cell locating plate according to <1>, wherein the coating liquid is a cell culture medium.
<8> The method for producing a cell locating plate according to <1>, wherein a pitch interval between the holes adjacent to each other is 300 micrometers or less.
<9> The method for producing a cell locating plate according to <1>, wherein a number of cells located in the holes is ±20% of an average value at more than or equal to 80% of the holes.
<10> A method for producing a member for containing cells, the method including:
laminating a structural layer over a plane of a base material, the structural layer being a layer that is formed of a cell non-adhesive material and in which a plurality of holes are formed;
attaching a cell adhesive material in the holes; and
coating a coating liquid in the holes and over the plane of the base material exposed through the holes.
<11> A member for containing cells, the member including:
a base material having at least a plane;
a structural layer over the plane, the structural layer being formed of a cell non-adhesive material and including a plurality of holes; and
a cell adhesive material attached in the holes.
<12> The member for containing cells according to <11>, the member further including
a coating liquid in the holes, the coating liquid coating the plane of the base material exposed through the holes.
<13> The member for containing cells according to <12>,
wherein the cell adhesive material is contactless with side surfaces of the holes.
<14> The member for containing cells according to <11>,
wherein the plane of the base material exposed through the holes in the structural layer is a surface onto which ink liquid droplets discharged by an inkjet method land.
<15> The member for containing cells according to <11>,
wherein a minimum thickness of the coating liquid is 5 micrometers or greater.
<16> The member for containing cells according to <11>,
wherein a pitch interval between the holes adjacent to each other is 300 micrometers or less.
<17> A cell locating plate including:
the member for containing cells according to <11>; and cells located in the plurality of holes of the member for containing cells.
<18> The cell locating plate according to <17>,
wherein a number of cells located in the holes is ±20% of an average value at more than or equal to 80% of the holes.
<19> The cell locating plate according to <17>,
wherein the cells located in the holes are the cells discharged by an inkjet method.
<20> The cell locating plate according to <17>,
wherein a number of kinds of cells located in the holes is two or more.

The method for producing a cell locating plate according to any one of <1> to <9>, the method for producing a member for containing cells according to <10>, a member for containing cells according to any one of <11> to <16>, and a cell locating plate according to any one of <17> to <20> can solve the various problems in the related art and can achieve the object of the present disclosure.

What is claimed is:

1. A method for producing a cell locating plate, the method comprising:
laminating a structural layer over a plane of a base material, the structural layer being a layer that is formed of a cell non-adhesive material and in which a plurality of holes are formed;
coating a coating liquid in the holes and over the plane of the base material exposed through the holes; and
discharging a cell suspension onto the plane coated with the coating liquid.

2. The method for producing a cell locating plate according to claim 1, the method further comprising
attaching a cell adhesive material in the holes.

3. The method for producing a cell locating plate according to claim 2, wherein the cell adhesive material is contactless with side surfaces of the holes.

4. The method for producing a cell locating plate according to claim 1, wherein the plane of the base material exposed through the holes in the structural layer is a surface onto which liquid droplets of the cell suspension discharged by an inkjet method land.

5. The method for producing a cell locating plate according to claim 1, wherein the cell non-adhesive material comprises a water-repellent material or a silicon-containing material.

6. The method for producing a cell locating plate according to claim 1, wherein a minimum thickness of the coating liquid is 5 micrometers or greater.

7. The method for producing a cell locating plate according to claim 1, wherein the coating liquid comprises a cell culture medium.

8. The method for producing a cell locating plate according to claim 1, wherein a pitch interval between the holes adjacent to each other is 300 micrometers or less.

9. The method for producing a cell locating plate according to claim 1, wherein a number of cells located in the holes is ±20% of an average value at more than or equal to 80% of the holes.

10. A method for producing a member for containing cells, the method comprising:
laminating a structural layer over a plane of a base material, the structural layer being a layer that is formed of a cell non-adhesive material and in which a plurality of holes are formed;
attaching a cell adhesive material in the holes; and
coating a coating liquid in the holes and over the plane of the base material exposed through the holes.

11. A member for containing cells, the member comprising:
a base material that comprises at least a plane;
a structural layer over the plane, wherein the structural layer is formed of a cell non-adhesive material and comprises a plurality of holes; and
a coating liquid in the holes, the coating liquid coating the plane of the base material exposed through the holes

12. The member for containing cells according to claim 11, further comprising
a cell adhesive material attached in the holes.

13. The member for containing cells according to claim 12,
wherein the cell adhesive material is contactless with side surfaces of the holes.

14. The member for containing cells according to claim 11,
wherein the plane of the base material exposed through the holes in the structural layer is a surface onto which ink liquid droplets discharged by an inkjet method land.

15. The member for containing cells according to claim 11,
wherein a minimum thickness of the coating liquid is 5 micrometers or greater.

16. The member for containing cells according to claim 11,
wherein a pitch interval between the holes adjacent to each other is 300 micrometers or less.

17. A cell locating plate comprising:
the member for containing cells according to claim 11; and
cells located in the plurality of holes of the member for containing cells.

18. The cell locating plate according to claim 17,
wherein a number of cells located in the holes is ±20% of an average value at more than or equal to 80% of the holes.

19. The cell locating plate according to claim 17,
wherein the cells located in the holes are the cells discharged by an inkjet method.

20. The cell locating plate according to claim 17,
wherein a number of kinds of cells located in the holes is two or more.
